(19) United States
(12) Patent Application Publication
(10) Pub. No.: US 2025/0095986 A1
(43) Pub. Date: Mar. 20, 2025

(54) TIN OXIDE NANORIBBON ACETONE GAS SENSOR, PREPARATION METHOD THEREOF AND USE THEREOF

(71) Applicant: Yangtze Delta Region Institute (Huzhou), University of Electronic Science and Technology of China, Huzhou City (CN)

(72) Inventors: Weiwu CHEN, Huzhou City (CN); Zhaojun QIN, Huzhou City (CN)

(21) Appl. No.: 18/493,894

(22) Filed: Oct. 25, 2023

(30) Foreign Application Priority Data
Sep. 15, 2023 (CN) 2023111971037

(51) Int. Cl. H01L 21/02; G01N 33/00

(52) U.S. Cl. CPC H01L 21/02565; G01N 33/0027; H01L 21/02628

(57) ABSTRACT

Disclosed is a method of preparing a tin oxide nanoribbon acetone gas sensor. The detailed steps are: placing tin oxide powder and silicon sheet into a horizontal tube furnace; pretreating gas inside the tube furnace; firing ultra-thin tin oxide nanoribbons at high temperature; dispersing ultra-thin tin oxide nanoribbons; preparing ultra-thin tin oxide nanoribbon acetone gas sensors. The tin oxide nanoribbons obtained by this preparation method are extremely thin, and the nanoribbons are flexible. The tin oxide nanoribbons deposited on the silicon sheet are easy to disperse, which brings greater convenience to the practical application of tin oxide nanoribbons. The tin oxide nanoribbons obtained according to this method of preparing ultra-thin tin oxide nanoribbons are sensitive to acetone gas, and therefore can be applied into an acetone gas sensor.

### CROSS-REFERENCE TO RELATED APPLICATIONS

This application claims priority to Chinese Patent Application No. 202311197103.7, filed on Sep. 15, 2023. The disclosure of the application is incorporated herein for all purposes by reference in its entirety.

### TECHNICAL FIELD

The present disclosure belongs to the field of toxic and harmful gas sensing and detection, and specifically relates to a tin oxide nanoribbon acetone gas sensor, a method of preparing the tin oxide nanoribbon acetone gas sensor and a use of the tin oxide nanoribbon acetone gas sensor in acetone gas detection.

### BACKGROUND

Nanomaterials have received widespread attention since their discovery. Among them, one-dimensional nanomaterials have a higher specific surface area than two-dimensional nanomaterials, which brings them stronger surface effects. For two-dimensional nanomaterials, a good planar structure makes them have fewer surface defects. As for nanoribbon materials, they can exhibit some of the advantages of one-dimensional and two-dimensional nanomaterials at the same time, so they have attracted increasing attention. Development of an ultra-thin nanoribbon material can further improve some features of the nanoribbon material, which facilitates its practical application.

Tin oxide is a semiconductor material, and a large number of experiments have proven that it has good performance on gas-sensing. Therefore, it is beneficial to the practical application of tin oxide materials in the field of gas sensing, especially to the application of flexible sensors, by developing a method for preparing ultra-thin tin oxide nanoribbons and enabling the nanoribbons to be used in the field of acetone gas detection. It is known that when oxide semiconductor materials are used for gas sensing applications, they are sensitive to target gases at temperatures of several hundred degrees Celsius; and tin oxide one-dimensional nanomaterials are sensitive to target gases at temperatures above two hundred degrees Celsius; therefore, people often dope rare earths and other precious metals to lower their working or response temperatures, which inevitably increases its cost.

The article “Flexible Acetone Gas Sensor based on ZIF-8/Polyacrylonitrile (PAN) Composite Film” published in the journal Acta Chimica Sinica points out that a wearable flexible gas sensor is a type of gas sensor with mobility and portability, and flexibility is an important factor. Therefore, the preparation of ultra-thin tin oxide nanoribbons and their sensitive response to acetone gas are of great significance for the development of flexible acetone gas sensors.

### SUMMARY

To overcome the above deficiencies in the prior art, present disclosure provides a tin oxide nanoribbon acetone gas sensor, preparation method and use same in acetone gas detection.

The technical solution adopted by the present disclosure is as follows:

A method for preparing a tin oxide nanoribbon acetone gas sensor comprising the steps of:
S1: placing high-purity tin oxide powder in a high-temperature area of a horizontal tube furnace, and placing a silicon sheet downstream from the tin oxide powder;
S2: bringing internal atmosphere of the tube furnace to a vacuum state, and then continuously introducing protective gas while transporting materials (i.e., heated tin oxide powder) until the preparation process is completed;
S3: heating the high-temperature area up to a predetermined temperature and maintaining it, cooling down the tube furnace naturally to obtain ultra-thin tin oxide nanoribbons attached to the silicon sheet;
S4: dispersing/providing/delivering the ultra-thin tin oxide nanoribbons on the silicon sheet into pure ethanol to obtain a suspension, dripping the suspension onto an additional clean silicon sheet in order to spread the ultra-thin tin oxide nanoribbons evenly on a surface of the silicon sheet;
S5: using a mask (the mask is a square copper ring, with regularly arranged tight grid of metal wires in the middle, any metal wire can be used, similar to the surface of a tennis racket) to cover the surface of the silicon sheet obtained in step S4, plating a metal film on the surface, and removing the mask to obtain a tin oxide nanoribbon acetone gas sensor.

In some embodiments, the silicon sheet is placed at a position of 15-20 cm downstream from the tin oxide powder in step S1.

In some embodiments, the tube furnace has a pressure of 20-50 Pa in the vacuum state, in step S2.

In some embodiments, the protective gas is introduced at a flow rate of 20-30 sccm, in step S2.

In some embodiments, the high temperature area is heated to 1300-1350° C. in step S3.

In some embodiments, the predetermined temperature is maintained for 110-120 min in step S3.

In some embodiments, the metal film has a thickness of 50-60 nm in step S5.

The beneficial effects of the present disclosure are as below:
(1) The equipments required and the preparation method by the disclosure are simple, and the raw materials are economical.
(2) The preparation method of the present disclosure can be used to obtain tin oxide nanoribbons, which have an extremely large width-to-thickness ratio and is flexible.
(3) The tin oxide nanoribbons obtained by the present disclosure have obvious structural advantages and are easily divided to make nanoribbon sensors. The obtained single tin oxide nanoribbon gas sensor can sensitively detect acetone gas at 160-180° C.

### BRIEF DESCRIPTION OF THE DRAWINGS

FIG. 1 is a transmission electron microscope image of an ultra-thin tin oxide nanoribbon.

FIG. 2 is a photo of a single ultra-thin tin oxide nanoribbon acetone gas sensor.

FIG. 3 is a gas sensitivity diagram of a single ultra-thin tin oxide nanoribbon acetone gas sensor.

FIG. 4 shows performance of a single ultra-thin tin oxide nanoribbon acetone gas sensor at different temperatures.

### DETAILED DESCRIPTION

The technical solutions in the embodiments of the present disclosure will be clearly and completely described below with reference to the accompanying drawings in the embodiments of the present disclosure. Obviously, the described embodiments are only some of the embodiments of the present disclosure, rather than all the embodiments. Based on the embodiments of the present disclosure, all other embodiments obtained by those of ordinary skill in the art without creative efforts fall within the scope of protection of the present disclosure.

### Example 1

A method of preparing a tin oxide nanoribbon acetone gas sensor includes steps of:
S1: placing high-purity tin oxide powder in a high-temperature area of a horizontal tube furnace, and placing a silicon sheet at a position of 20 cm downstream the tin oxide powder,
S2: bringing internal atmosphere of the tube furnace to a vacuum state of about 40 Pa by a mechanical pump, and then continuously introducing high pure argon at a flow rate of 30 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet such that the ultra-thin tin oxide nanoribbons are evenly arranged on a surface of the silicon sheet;
S5: using a mask with a grid (grid wire has a diameter of 15 μm) to cover the surface of the silicon sheet obtained in step S4, plating a gold film with a thickness of 60 nm on the surface, and removing the mask to obtain a single tin oxide nanoribbon acetone gas sensor.

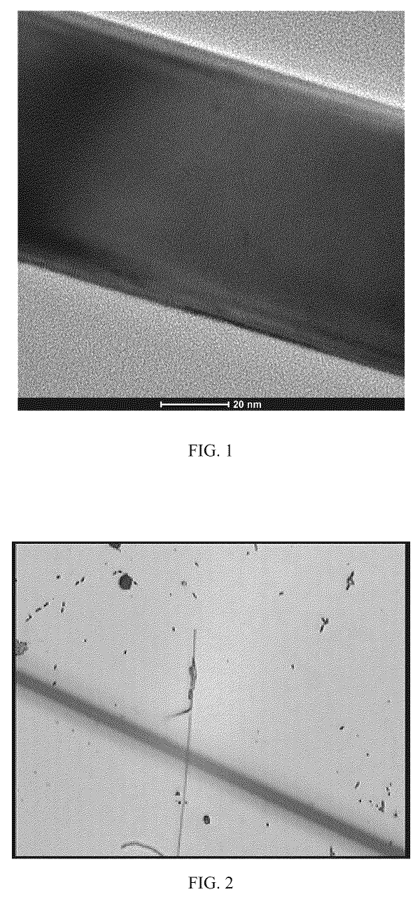

The tin oxide nanoribbons obtainable in this example have an extremely large width-to-thickness ratio, with a width of 90 nm and a thickness of 5 nm. The transmission electron microscope image is shown in FIG. 1. The photo of the obtained single ultra-thin tin oxide nanoribbon gas sensing device (see FIG. 2) can be obtained by an optical microscope.

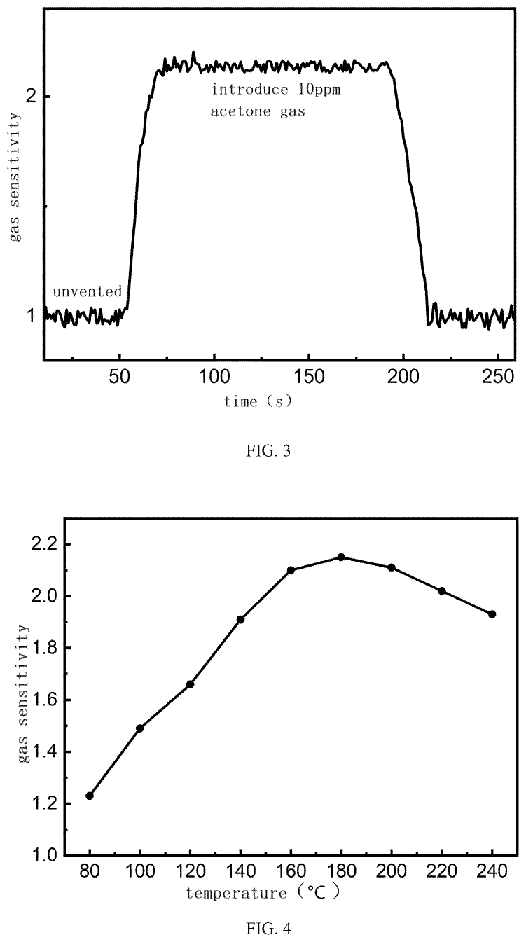

The single ultra-thin tin oxide nanoribbon gas sensor device prepared in this example is connected to a current detector. By placing the sensor in acetone gas environment, the detector reports significant current fluctuations (see FIG. 3), indicating that the sensor is sensitive to acetone gas. Moreover, as shown in FIG. 4, by controlling the temperature of the sensor through a heating platform, it is found that a single ultra-thin tin oxide nanoribbon can achieve 2.15 times the magnitude of the current change at 180° C. for 10 ppm ultra-low concentration of acetone gas and even if the temperature decreases to 160° C., it can still exhibit 2.1 times the magnitude to 10 ppm acetone concentration.

### Example 2

A method of preparing a tin oxide nanoribbon acetone gas sensor includes steps of:
S1: placing high-purity tin oxide powder in a high-temperature area of a horizontal tube furnace, and placing a silicon sheet at a position of 16 cm downstream the tin oxide powder;
S2: bringing internal atmosphere of the tube furnace to a vacuum state of 20 Pa by a mechanical pump, and then continuously introducing high purity nitrogen at a flow rate of 23 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet in order to spread the ultra-thin tin oxide nanoribbons evenly on a surface of the silicon sheet;
S5: using a mask with a grid (grid wire has a diameter of 11 μm) to cover the surface of the silicon sheet obtained in step S4, plating a titanium film with a thickness of 52 nm on the surface, and removing the mask to obtain a single tin oxide nanoribbon acetone gas sensor.

### Example 3

A method of preparing a tin oxide nanoribbon acetone gas sensor includes steps of:
S1: placing high-purity tin oxide powder in a high-temperature area of a horizontal tube furnace, and placing a silicon sheet at a position of 15 cm downstream from the tin oxide powder;
S2: bringing internal atmosphere of the tube furnace to a vacuum state of 25 Pa by a mechanical pump, and then continuously introducing high pure argon at a flow rate of 24 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet in order to spread the ultra-thin tin oxide nanoribbons evenly on a surface of the silicon sheet;
S5: using a mask with a grid (grid wire has a diameter of 12 μm) to cover the surface of the silicon sheet obtained in step S4, plating a copper film with a thickness of 55 nm on the surface, and removing the mask to obtain a single tin oxide nanoribbon acetone gas sensor.

### Example 4

A method of preparing a tin oxide nanoribbon acetone gas sensor includes steps of:
S1: placing high-purity tin oxide powder in a high-temperature area of a horizontal tube furnace, and placing a silicon sheet at a position of 18 cm downstream from the tin oxide powder;
S2: bringing internal atmosphere of the tube furnace to a vacuum state of 30 Pa by a mechanical pump, and then continuously introducing high pure argon at a flow rate of 26 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet in order to spread the ultra-thin tin oxide nanoribbons evenly on a surface of the silicon sheet;
S5: using a mask with a grid (grid wire has a diameter of 13 μm) to cover the surface of the silicon sheet obtained in step S4, plating a gold film with a thickness of 57 nm on the surface, and removing the mask to obtain a single tin oxide nanoribbon acetone gas sensor.

### Example 5

A method of preparing a tin oxide nanoribbon acetone gas sensor includes steps of:
S1: placing high-purity tin oxide powder in a high-temperature area of a horizontal tube furnace, and placing a silicon sheet at a position of 18 cm downstream from the tin oxide powder;
S2: bringing internal atmosphere of the tube furnace to a vacuum state of 35 Pa by a mechanical pump, and then continuously introducing high purity nitrogen at a flow rate of 25 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet in order to spread the ultra-thin tin oxide nanoribbons evenly on a surface of the silicon sheet,
S5: using a mask with a grid (grid wire has a diameter of 12 μm) to cover the surface of the silicon sheet obtained in step S4, plating a titanium film with a thickness of 56 nm on the surface, and removing the mask to obtain a single tin oxide nanoribbon acetone gas sensor.

### Example 6

A method of preparing a tin oxide nanoribbon acetone gas sensor includes steps of:
S1: placing high-purity tin oxide powder in a high-temperature area of a horizontal tube furnace, and placing a silicon sheet at a position of 19 cm downstream from the tin oxide powder;
S2: bringing internal atmosphere of the tube furnace to a vacuum state of 42 Pa by a mechanical pump, and then continuously introducing high purity nitrogen at a flow rate of 28 sccm while transporting materials until the preparation process is completed;
S3: after heating up the high-temperature area to 1350° C. and maintaining the temperature for 120 min, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet in order to spread the ultra-thin tin oxide nanoribbons evenly on a surface of the silicon sheet;
S5: using a mask with a grid (grid wire has a diameter of 15 μm) to cover the surface of the silicon sheet obtained in step S4, plating a titanium film with a thickness of 58 nm on the surface, and removing the mask to obtain a single tin oxide nanoribbon acetone gas sensor.

Although the specific embodiments of the present disclosure have been described in detail in connection with embodiments, they should not be understood as limiting the scope of the disclosure. Within the scope described in the claims, various modifications and transformations that can be made by those skilled in the art without creative work still fall within the scope of protection of this disclosure.

1. A method of preparing a tin oxide nanoribbon acetone gas sensor, comprising the steps of:
S1: placing high-purity tin oxide powder in a controlled-temperature area of a horizontal tube furnace, and placing a silicon sheet downstream the tin oxide powder along a flow path within the tube furnace;
S2: providing internal atmosphere of the tube furnace to a vacuum state, and then continuously introducing protective gas while transporting materials until the preparation process is completed;
S3: heating the controlled-temperature area to a predetermined temperature and maintaining it, cooling down the tube furnace naturally to obtain ultra-thin tin oxide nanoribbons attached to the silicon sheet;
S4: dispersing the ultra-thin tin oxide nanoribbons on the silicon sheet into pure ethanol to obtain a suspension, dripping the suspension onto a clean silicon sheet such that the ultra-thin tin oxide nanoribbons are arranged evenly on a surface of the silicon sheet;
S5: covering the surface of the silicon sheet obtained in step S4 with a mask, plating a metal film on the surface of the silicon sheet, and removing the mask to obtain a tin oxide nanoribbon acetone gas sensor.

2. The method according to claim 1, wherein the silicon sheet is placed at a position 15-20 cm downstream the tin oxide powder in step S1.

3. The method according to claim 1, wherein the tube furnace, in the vacuum state, has a pressure of 20-50 Pa in step S2.

4. The method according to claim 1, wherein the protective gas is introduced at a flow rate of 20-30 sccm in step S2.

5. The method according to claim 1, wherein the high temperature area is heated to 1300-1350° C. in step S3.

6. The method according to claim 1, wherein the predetermined temperature is maintained for 110-120 min in step S3.

7. The method according to claim 1, wherein the mask has a grid with a grid wire diameter of 10-15 μm in step S5.

8. The method according to claim 1, wherein the metal film has a thickness of 50-60 nm in step S5.

9. A tin oxide nanoribbon acetone gas sensor prepared according to the method of claim 1.

10. Use of the tin oxide nanoribbon acetone gas sensor according to claim 9 in acetone gas detection.
